(19) United States
(12) Patent Application Publication
(10) Pub. No.: US 2019/0365454 A1
(43) Pub. Date: Dec. 5, 2019

(54) Enhanced Thermal Pusher Assembly

(71) Applicant: BALT USA, Irvine, GA (US)

(72) Inventors: Jake Le, Foothill Ranch, CA (US); David Ferrera, Goto de Gaza, CA (US); Randall Takahashi, Mission Viejo, CA (US); Dawson Le, Garden Grove, CA (US)

(21) Appl. No.: 16/476,524

(22) PCT Filed: Jan. 8, 2018
(86) PCT No.: PCT/US2018/012838

§ 371 Date: Jul. 8, 2019

(51) Int. Cl. A61B 18/08

(52) U.S. Cl. CPC A61B 18/082; A61B 2018/00404

(57) ABSTRACT

Enhanced thermal pusher assembly ensures: proper delivery of coils profiled for various applications without grounding by thermal attachment and with a handheld controller, in under one second. Attachment controller configures interface with Al, —interface and data transmission and management system.

### FIELD

The present disclosures relate to the field of neurovascular technologies. Specifically, the present disclosure teaches novel pushing means for performing endovascular procedures within the brain, although the devices of the instant invention are easily modified for other medical device applications. Devices embodied in this description currently include the OPTIMA® lines of endovascular coils (BALT USA, Irvine, Calif. 92618)

### BACKGROUND OF THE DISCLOSURES

Those skilled in the art and those whom they operate on have increased geometrically in number. Accordingly, techniques and devices to make their respective brains more easily serviced have become important and needing to be improved.

Large medical device companies have expended a tremendous volume of resources to more easily access the afflicted brain.

Unfortunately, step-wise improvements need to be called out each time that an easier way to put, for example, coils into an aneurysm in the brain arises.

### OBJECTS AND SUMMARY OF THE INVENTION

Briefly stated, enhanced thermal pusher assembly ensures proper delivery of coils profiled for various applications without grounding by thermal attachment and with a handheld controller.

According to embodiments, there is provided an improved thermal coil medical device system which comprises, in combination; a full line of platinum coils ranging from an array of features, sizes and designs; whereby a predetermined size, length and softness profile is required:

and at least one coil is delivered without grounding by thermal detachment in conjunction with a handheld controller.

According to embodiments, there is provided a method of manufacturing an implant thermal pusher assembly which comprises, in combination:

crafting a subassembly further comprising the steps of, inserting lead wireshrinking a PET jacket over a body coil;

and centering said PET without burning it or creating holes over its length;

whereby the distal end of the PET jacket begins within 1-2 revolutions of the proximal end of the gapped section of the body coil, and forms a unique positive contrast radiographically viewable zone.

According to embodiments, there are provided Products, by the processes herein whereby resistance between the connector and hypotube is between 36-53 OHMS, and further comprising positive contrast warning stripe PET jackets without any inherent surface aberrations, distortions or other aspects that could impact the vessels of a patient.

### BRIEF DESCRIPTION OF THE DRAWINGS

Various preferred embodiments are described herein with references to the drawings in which merely illustrative views are offered for consideration, whereby:

FIG. 1 is a cartoon/schematic of a version of the present inventions;

FIG. 2 is a cartoon/schematic of a version of the present inventions;

FIG. 3 is cartoon/schematic of a version of the present inventions;

FIG. 4 is a cartoon/schematic of a version of the present inventions;

FIG. 5 is cartoon/schematic of a version of the present inventions;

FIG. 6 is a cartoon/schematic of a version of the present inventions;

FIG. 7 is a cartoon/schematic of a version of the present inventions;

FIG. 8 is a cartoon/schematic of a version of the present inventions.

### DETAILED DESCRIPTIONS

The present inventors have optimized a pushing means for delivery of brain coils. For guidance only are the following references, each of which as of the fourth quarter update searching done have showed the following list to only define the state of the art and not impact the instant teachings:

U.S. Pat. Nos. 4,346,712; 5,059,213; 5,108,407; 5,122,136; 5,217,484; 5,354,245; 5,382.259; 5,470,338; 5,498,250; 5,540,680; 9,414.819; 5,582,619; 5,759,161; 5,853.418; 5,925,037; 6,010,498 C1 & C2; 6,066,133; 6,238,145; 6,277,126; 6,338,657; 6,478,773; 6,500,149; 6,966,892

Novel enhanced and unique pushing members constitute elements of a system which comprises a coil or implant delivery system also driven by a proprietary hand controller which drives a conductive member specialized insulating and conductive layers and an effective means for communicating with power sources and shown and described below, and claimed and shown in the appendix.

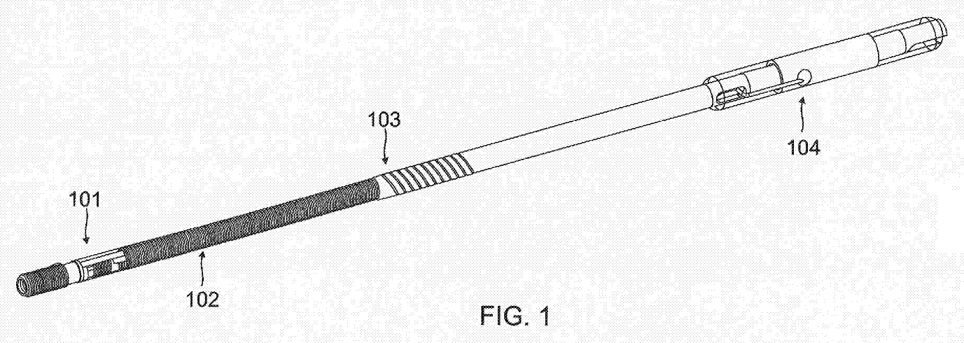

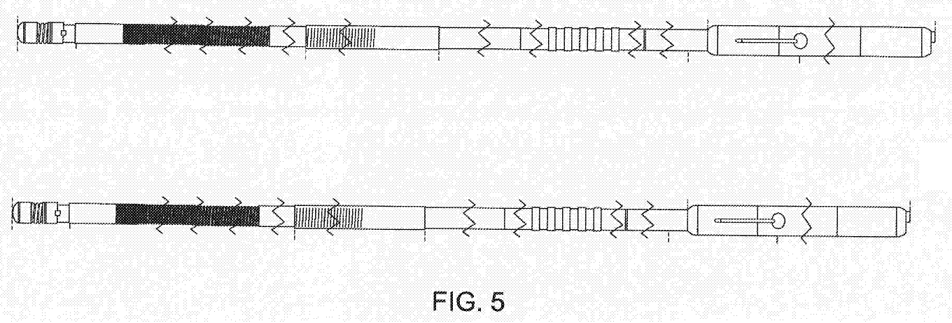

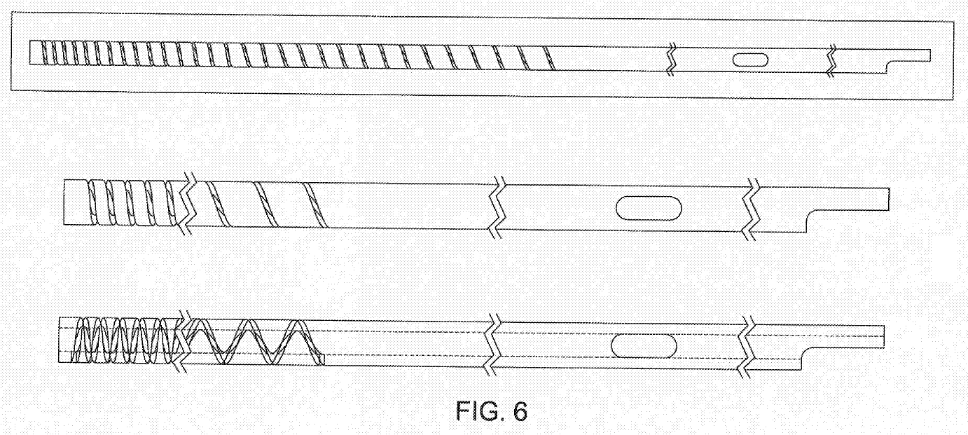

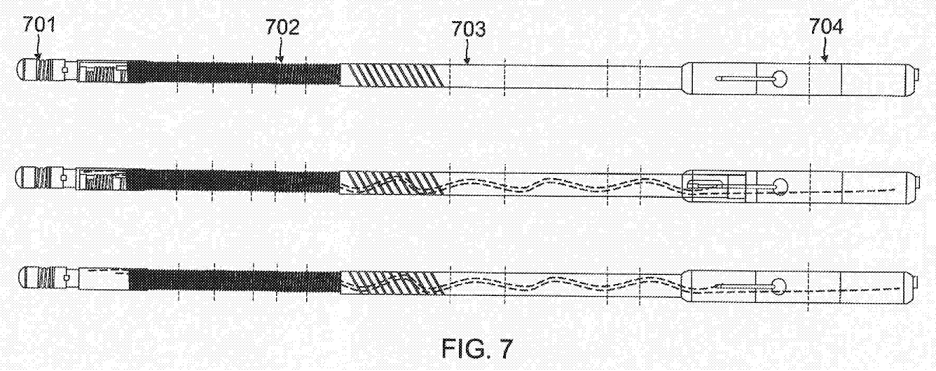

Referring now to the FIGS. 1-8, which summarize and schematize the subject matters of the present invention, FIG. 1 is a cartoon/schematic depiction wherein the customer-driven data needed for this system have been embodied. Namely USERS being Physicians have dictated a number of structural changes so that the system feels better subjectively to access and maneuver in the tightly constrained space of the cerebral vasculature.

As shown, the instant thermal detachment system features a distal body and proximal hypotube, so that the device can be forcibly delivered, yet have the “hand/feel” associated with the subject implants or coils themselves (like a fly-fishing lure in a mountain stream, according to a practitioner). These features are coupled with desiderata of instantaneous and reliable detachment via a single use handheld controller. In terms of exclusionary embodiments, those vestiges of the older electrolytic detachment systems which those skilled in the art have now switch from are likewise listed. Instant detachment at end 101 is less than 9 second, soft body coil 102 is featured for stability and to prevent microcatheter lock-out.

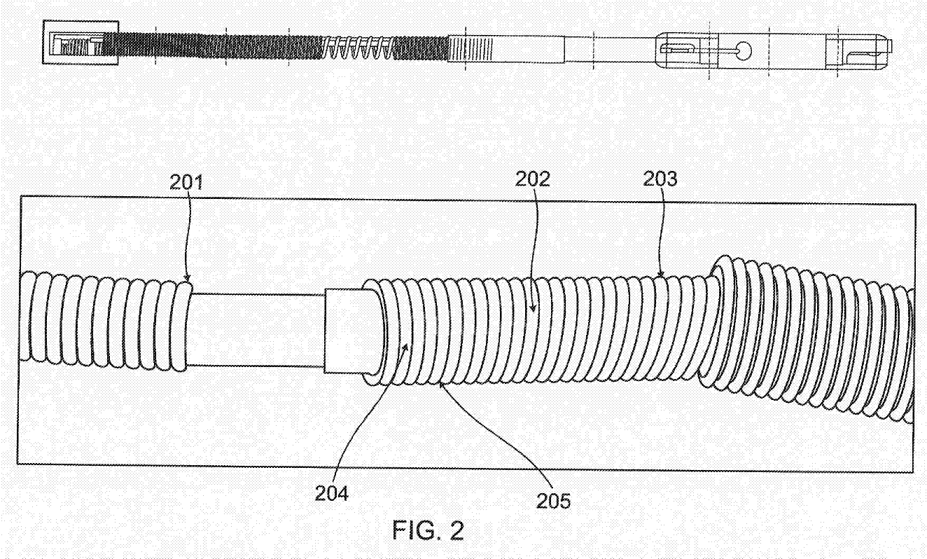

Referring also to FIG. 2-8 further depict detailed structural make-up of the system, whereby in manufacturing, for example, one attaches green and yellow lead wires disposed under PET jackets such that the proximal end of the PET jackets, including PET for body coil and under connector are adjacent to and contiguous with the hypotube to body assembly making sure that the insulation PET remains centered between hole and notch.

FIG. 2 is a design magnification summary showing implant 201 heater coil pinch 204, heater coil 202, lead wires 203 and heat shield 205. Single Gold connector, 104, is highly efficient. The other figures continue to show the solder heater coil, lead wires and epoxy-gold connector (in this embodiment and not meant to be limiting) heater coil is shown in relation to PET for strain relief and in detail with the Liquid Polyimide and Polyimide shield shown proximate to Dymax adhesive and showing where the yellow lead wires is soldered at the near end of said heater coil for the OPTIMA® systems (intra).

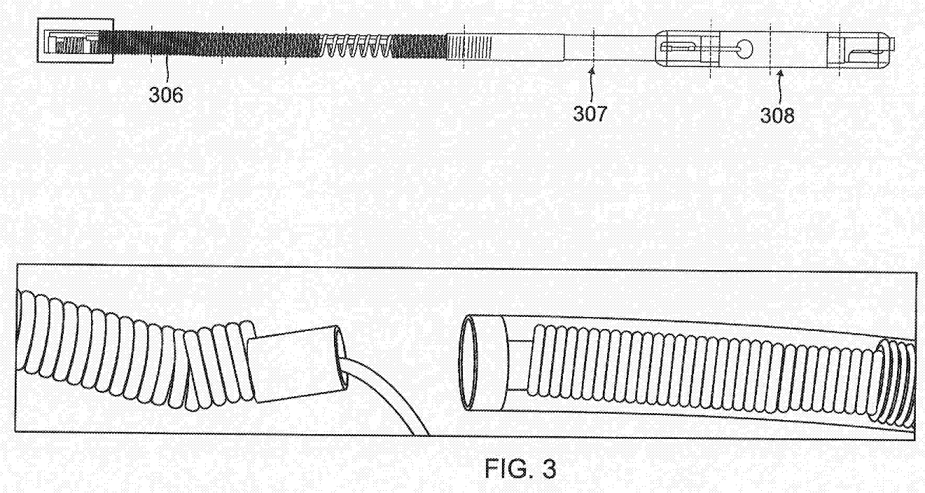

FIGS. 3-8 starting with FIG. 3, show body coil 306, hypotube body 307 and single gold connector 308 with cartoons juxtaposed with digital imagery.

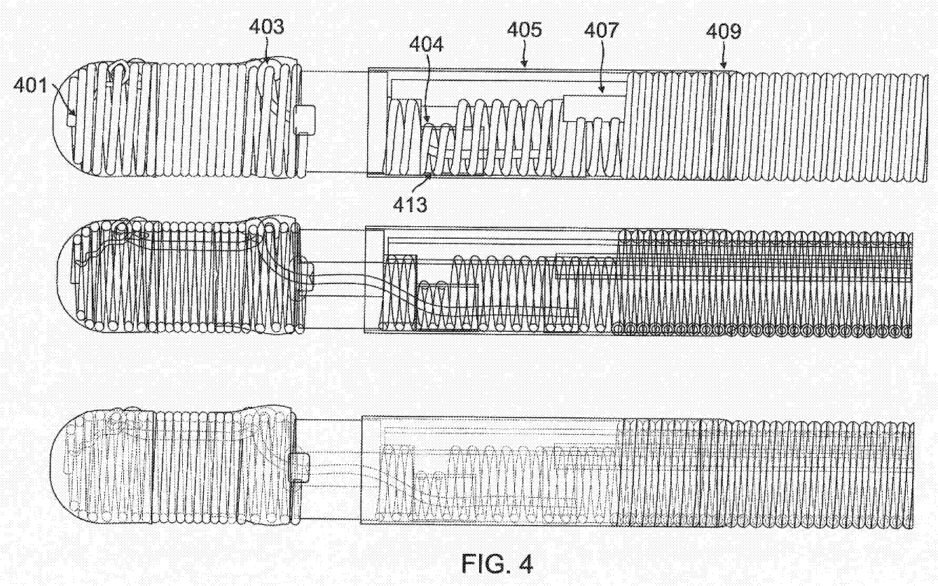

FIG. 4 is a side profile implant 401, SR thread 403, pinched heat coil 404 and heater coil 405.

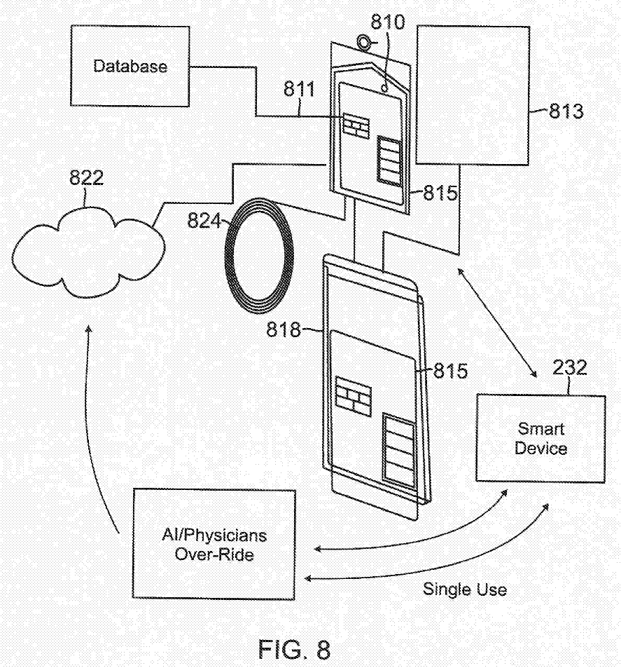

FIG. 8 are cartoons and schematics with no reference to scale for the enhanced hand held thermal attachment controllers, unique to the instant system 822, including mechanical and electronic elements 810-824, which comprise 811 upper surface and 818 lower surface, chipsets, wires 824, clouds and databases that interface by homunculi 232-236 with Artificial Intelligence and stored patient records. System 822 in optimal form is a disposable that allows for detachment of distal body coil in a very soft hand way without compromising the robust pushability. Thermal detachment with the Optima® System allows handheld single use to benefit the physical with all detachment control Alternately, linkage to any databases or systems to manage patient information, 232, 254, 234 (clouds) and 236 are shown. No electrode or groin needle, IV pole or cables are needed to detach under a second.

The resultant medical device system is able to be navigated better than competing systems to deliver and release the most coils reliably, which providing the requisite “hand/feel” required by the best practitioners. As opposed to other who claim to use further mechanism to drive release of the coils—the instant system as configured enable reliable and consistent delivery, and is made by A method of manufacturing an implant delivery system comprising, in combination—the steps of; providing at least a pusher component, which comprises, in combination: at least a tether defining a first end and a second end, and an implant component; said method comprising the steps of, attaching the first end of said at least a tether to a first component of said implant delivery system, placing a portion of the first component in proximity to a portion of a second component (to ensure that the implant is touching the pusher before extending the tether), extending said at least a tether to a predetermined distance (for example from at least about 0.200″ to twice or three times that length) attaching said second end of said at least a tether to the second component (by adhesion or gluing the at least a tether to the pusher prior to knotting the tether to the pusher).

Likewise, those skilled in the art readily understand that said first component is said the pusher and the second component the implant.

Different from the art is the instant method of manufacturing an implant delivery system by providing at least a pusher component, which comprises, in combination:

at least a tether defining a first end and a second end, and an implant component;

said method comprising the steps of, attaching the first end of said at least a tether to a first component of said implant delivery system, by placing a portion of the first component in proximity to a portion of a second component (to ensure that the implant is touching the pusher before extending the tether); extending said at least a tether to a predetermined distance (for example from at least about 0.200″ to twice or three times that length).

and attaching said second end of said at least a tether to the second component (by adhesion or gluing the at least a tether to the pusher prior to knotting the tether to the pusher).

Since the physicians know how the coil feels when pushed and become accustomed to the same, it is respectfully proposed that this “hand/feel” is best replicated whereby the movement of the system, in situ, is created without having pre-tensioned/post-tensioned or other aspects included with the coils to be delivered. That, it is respectfully submitted, is the talc told by the Prior Art reviewed in light of Dr. input and clinical results.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined. i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language mans that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. An improved thermal coil medical device system which comprises, in combination;
a full line of platinum coils ranging from an array of features, sizes and designs;
whereby a predetermined size, length and softness profile is required;
and at least one coil is delivered without grounding by thermal detachment in conjunction with a handheld controller, in less than one second.

2. The system of claim 1, for use within the neurovasculature to endovascularly obstruct blood flow to address at least one of intracranial aneurysms, arteriovenous malformations, or arterial and venas embolized aspects of the peripheral vasculature.

3. The system of claim 2, further comprising a purely physician controlled operating sequence and a distal body coil;
a proximal spiral laser cut hypotube (this spiral laser cut allows for improved flexibility and deliverability. The spiral laser cut is located at the junction of the body coil to hypotube to balance the flexibility transition);
thermal detachment; and
a custom detachment controller which is handheld and for single use.

4. The System of claim 3, whereby the distal body coil is extremely soft, provides no undesired kickback and provides the hand or feel desired by a practitioner to feel coil-like, to facilitate consistent delivery by physicians.

5. The System of claim 4, providing instantaneous and reliable detachment, independent of patient physiology of morphology; no MR artifact (no stainless steel implanted) and no return electrode or groin needles, cables or IV pole mounted controller.

6. A method of manufacturing an implant thermal pusher assembly which comprises, in combination:
crafting a subassembly further comprising the steps of:
inserting lead wires; such that there is excess lead wire length inside the pusher assembly to create slack of the leadwires within the pusher;
shrinking a PET jacket over a body coil;
and centering said PET without burning it or creating holes over its length;
whereby the distal end of the PET jacket begins within 1-2 revolutions of the proximal end of the gapped section of the body coil, and forms a unique positive contrast radiographically viewable zone.

7. Products, by the process of claim 6 whereby resistance between the connector and hypotube is between at least about 36 and 53 OHMS;
and further comprising positive contrast warning stripe PET jackets without any inherent surface aberrations, distortions or other aspects that could impact the vessels of a patient.

8. A method of manufacturing an implant delivery system comprising:
Providing at least a pusher component, which comprises, in combination: at least a tether defining a first end and a second end, and an implant component; said method comprising the steps of:
a. attaching the first end of said at least a tether to a first component of said implant delivery system;
b. placing a portion of the first component in proximity to a portion of a second component (to ensure that the implant is touching the pusher before extending the tether);
c. extending said at least a tether to a predetermined distance (for example from at least about 0.200″ to twice or three times that length)
d. attaching said second end of said at least a tether to the second component (by adhesion or gluing the at least a tether to the pusher prior to knotting the tether to the pusher).

9. The method of claim 8, wherein said first component is said implant component and said second component is said delivery pusher component.

10. The method of claim 8, wherein said first component is said delivery pusher component and said second component is said implant component.

11. The method of claim 10, wherein said at least a tether is attached near said proximal end of said coil.

12. The method of claim 10, wherein said at least a tether passes substantially through inner lumen of said coil and is attached near said distal end of said coil.

13. The method of claim 13, wherein said implant component is not a stent.

14. The system of claim 1, further comprising a shock absorbing means-for-spacing of round and wound coils.

15. The system of claim 14, wherein improved navigability of tortious vessels happens.

16. The system of claim 15, separable into multiple coils.

17. The system of claim 16, said multiple is two.

18. The system of claim 17, said multiple is greater than two.

19. The system of claim 18, said multiple is between 3 and 7.
